(19) United States
(12) Patent Application Publication
(10) Pub. No.: US 2005/0239723 A1
(43) Pub. Date: Oct. 27, 2005

(54) Compositions and methods useful for treatment of acne

(76) Inventors: Avinash N. Amin, St. Louis, MO (US); Michael Douglas, St. Louis, MO (US)

Correspondence Address: THOMPSON COBURN, LLP, ONE US BANK PLAZA, SUITE 3500, ST LOUIS, MO, 63101, US

(21) Appl. No.: 11/086,551

(22) Filed: Mar. 22, 2005

Related U.S. Application Data
(60) Provisional application No. 60/565,566, filed on Apr. 27, 2004.

(51) Int. Cl. A61K 31/7048; A61K 31/455; A61K 31/203; A61K 31/192

(52) U.S. Cl. 514/28; 514/355; 514/154; 514/29; 514/568; 514/400; 514/559; 514/725

(57) ABSTRACT

Methods and compositions related to treating, controlling or inhibiting acne vulgaris by reducing or inhibiting growth of Proprionibacterium acnes employing a compound having the following structure: 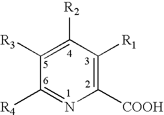 or a pharmaceutically acceptable salt thereof,
wherein R₁, R₂, R₃ and R₄ are selected from a group consisting of a carboxyl group, methyl group, ethyl group, propyl group, isopropyl group, butyl group, isobutyl group, secondary butyl group, tertiary butyl group, pentyl group, isopentyl group, neopentyl group, fluorine, chlorine, bromine, iodine and hydrogen.

### CROSS REFERENCE TO RELATED APPLICATIONS

This application claims priority from U.S. provisional application No. 60/565,566 filed on Apr. 27, 2004.

### FIELD OF THE INVENTION

The present invention relates to compositions and methods for treatment or control of acne.

### BACKGROUND OF INVENTION

Acne vulgaris is the most common cutaneous disorder. Propionibacterium acnes is the predominant microorganism present in acne. Sebaceous follicles involved in acne are characterized by the accumulation of abnormally desquamated comeocytes and excess sebum—the microcomedo. (Leyden, James L., The Evolving Role of Proprionibacterium Acnes in Acne, SEMINARS IN CUTANEOUS MEDICINE AND SURGERY, 20(3):139-143, Sep. 2001). This environment provides ideal growth conditions for P. acnes. Several orders of magnitude level of P. acnes are found in microcomedos. Levels of P. acnes colonization are highest in areas that are rich in sebaceous glands such as the scalp and face.

Acne vulgaris affects approximately 115 million patients in Europe, the United States, and Japan. The first line therapy for mild to moderate acne vulgaris patients includes topical antibiotics and retinoids. These therapies are all limited by several factors including topical irritation, photo-sensitization, antimicrobial resistance, and marginal efficacy. For antibiotics, systemic tetracycline, doxycycline, clindamycin, erythromycin, azithromycin, minocycline, and others, and topical versions of these and others have been used to treat acne. However, in the last decade, decreased sensitivity to antibiotics has developed and clinical resistance has been reported. Particularly, decreased sensitivity of P. acnes to antibiotics has developed, especially in patients treated for prolonged periods with antibiotics, with resulting decreased clinical benefit.

Tretinoin is currently accepted to be one of the most effective topical agents on the market. However, it reduces total lesions counts by only 32-45% and is photosensitizing. Moreover, tretinoin can cause both skin irritation and blistering.

Picolinic acid is a metabolite of tryptophan and is produced via amino acid breakdown in vivo. It affects zinc binding within zinc finger proteins. Picolinic acid has been generally reported to affect ion traffic (Evans, G. W., and P. E. Johnson, Characterization and quantitation of a zinc-binding ligand in human milk. PEDIATRI. RES., 14:876-880, 1980; Fernandez-Pol, J. A., Transition metal ions induce cell growth in NRK cells synchronized in G1 by picolinic acid, BIOCHEM. BIOPHYS. RES. CUMMUN., 76:413-419, 1977), cell cycle (Fernandez-Pol J A. Iron: possible cause of the G₁ arrest induced in NRK cells by picolinic acid. BIOCHEM. BIOPHYS. RES. COMMUN., 78(1):136-143, Sep. 9, 1977; Johnson, G. S. and J. A. Fernandez-Pol, NRK cells synchronized in G₁ by picolinic acid are super-sensitive to prostaglandin E1 stimulation, FEBS. LETT., 74(2):201-204, Mar. 1, 1977), bacterial growth (Collins J. J. et al., Transient growth inhibition of Escherichia coli K-12 by ion chelators: “in vivo” inhibition of ribonucleic acid synthesis, J. BACTERIOL., 138(3):923-932, 1979; Fortnagel, P. and E. Freese, Inhibition of aconitase by chelation of transition metals causing inhibition of sporulation in Bacillus subtilis, J. BIOL. CHEM., 243(20):5289-5295, Oct. 25, 1968), and host immune response (Ruffmann R, et al., In vivo activation of macrophages but not natural killer cells by picolinic acid (PLA), J. IMMUNOPHARMACOL., 6(4):291-304, 1984; Varesio L. et al., Picolinic acid, a catabolite of tryptophan, as the second signal in the activation of IFN-gamma-primed macrophages, J. IMMUNOL., 145(12):4265-4271, Dec. 15, 1990). Picolinic acid may also possess antitumor effects in vivo. A study showed that tumor-bearing mice showed reduced tumor size and augmented median survival time upon treatment with this agent. Blasi, E. et al., Protective Effect of Picolinic Acid on Mice Intracerebrally Infected with Lethal Doses of Candida albicans, ANTIMICROBIAL AGENTS AND CHEMOTHERAPY, 37(11):2422-2426, November 1993.

The mechanism by which picolinic acid exerts its effect is not entirely elucidated. One study shows the effect of picolinic acid (pyridine-2-carboxylic acid) on the efflux of divalent metal ions from multilamellar liposomes. Extraliposomal picolinic acid increased the efflux of Zn, Cu, Co, Mn, Ni, Cd, Pb, Fe(II) and Ca from the vesicles. However, when picolinic acid was trapped with Co, Cu and Zn within the liposomes, the loss of metals was reduced. In a partition study, picolinic acid increased the aqueous solubility of Zn, Cu, Co and Cd at alkaline pH, but did not transfer the metal to an organic bulk phase of chloroform. It has been hypothesized that picolinic acid does not act as an ionophore and that any effect it may have on zinc metabolism is dependent upon its unselective chelating properties, which may also lead to altered dietary and systemic compartmentation of other divalent cations. Aggett, P. J., An in vitro study of the effect of picolinic acid on metal translocation across lipid bilayers, J. NUTR., 119(10):1432-7, October 1989.

Acne afflicts millions of people worldwide. Current available therapies have a variety of disadvantages, ranging from adverse effects (blistering, photosensitivity, allergic reactions, etc.) in patients to a lack of or minimal effectiveness in patients (e.g. due to microbial resistance to the therapeutic agents). Accordingly, there continues to be need for an alternative therapeutic means for treating or controlling acne, particularly acne vulgaris.

### SUMMARY OF INVENTION

The inventors have determined that topical administration of a composition comprising picolinic acid, or derivatives thereof, is effective in controlling or treating acne.

The present invention provides a pharmaceutical composition comprising a compound having the following structure: 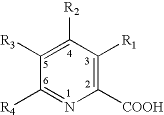
or a pharmaceutically acceptable salt thereof, wherein R₁, R₂, R₃ and R₄ are selected from a group consisting of a carboxyl group, methyl group, ethyl group, propyl group, isopropyl group, butyl group, isobutyl group, secondary butyl group, tertiary butyl group, pentyl group, isopentyl group, neopentyl group, fluorine, chlorine, bromine, iodine and hydrogen, and wherein the amount of the compound in the pharmaceutical composition is sufficient to reduce or inhibit growth of Proprionibacterium acnes by at least 1 or 2 logarithm.

The present invention further provides a method for treating or controlling acne vulgaris comprising administering to a subject afflicted with acne vulgaris a therapeutically effective amount of a composition comprising a compound having the following structure: 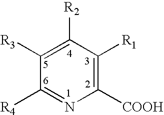
or a pharmaceutically acceptable salt thereof,

wherein R₁, R₂, R₃ and R₄ are selected from a group consisting of a carboxyl group, methyl group, ethyl group, propyl group, isopropyl group, butyl group, isobutyl group, secondary butyl group, tertiary butyl group, pentyl group, isopentyl group, neopentyl group, fluorine, chlorine, bromine, iodine and hydrogen, and wherein the composition reduces or inhibits growth of Proprionibacterium acnes.

### BRIEF DESCRIPTION OF THE DRAWINGS

FIG. 1 shows the results of the mean lesion counts per protocol analysis. A reduction of 58.2%, 55.5%, and 59.7% in mean total, inflammatory, and noninflammatory lesion counts, respectively, was observed using per protocol analysis. The results were from a study of twenty patients (5 males, 15 females) who were enrolled with varying ethnicity: Caucasian(12), African American (6), Hispanic (1), and Asian (1). The age range was from 20 to 48 years (mean 29.6 years). All subjects received open-label PCL-016 10% gel to be applied twice daily to the face over 12 weeks.

FIG. 2 shows a reduction of 50.6%, 47.2%, and 52.4%, respectively, in an intent-to-treat analysis. The experiment was performed as indicated for the per protocol analysis described under FIG. 1 above.

### DESCRIPTION OF THE INVENTION

Propionibacterium acnes (P. acnes) is the most common gram-positive microaerophilic organism found on normal skin. Although it has no intrinsic pathogenicity, P. acnes is believed to play a major role in the pathogenesis of acne. Most presently available topical anti-acne preparations such as benzoyl peroxides and topical antimicrobials exert their therapeutic effect through inhibition of P. acnes in vivo as demonstrated by a 1.0 to 2.0 logarithmic colony reduction.

Picolinic acid and/or its derivatives offer an alternative to controlling or treating acne. Picolinic acid and its derivatives were described in U.S. Pat. No. 6,743,771 B2, filed on Jul. 12, 2001, and is hereby incorporated by reference. Picolinic acid and its derivatives is represented by the following structure: 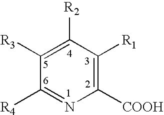
wherein R₁, R₂, R₃ and R₄ are selected from the group consisting of a carboxyl group, methyl group, ethyl group, propyl group, isopropyl group, butyl group, isobutyl group, secondary butyl group, tertiary butyl group, pentyl group, isopentyl group, neopentyl group, fluorine, chlorine, bromine, iodine and hydrogen.

The present invention provides a pharmaceutical compositions for controlling or treating acne vulgaris comprising a compound having the following structure: 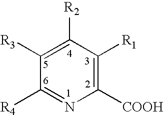
or a pharmaceutically acceptable salt thereof, wherein R₁, R₂, R₃ and R₄ are selected from a group consisting of a carboxyl group, methyl group, ethyl group, propyl group, isopropyl group, butyl group, isobutyl group, secondary butyl group, tertiary butyl group, pentyl group, isopentyl group, neopentyl group, fluorine, chlorine, bromine, iodine and hydrogen, and wherein the composition reduces or inhibits growth of Proprionibacterium acnes by at least 1 to 2 logarithm.

In one embodiment, the composition further comprises propylene glycol, ethyl alcohol, hydroxyethyl cellulose, sodium chloride, and water. Preferably, R₃ of the compound is a butyl group. More preferably, the compound is picolinic acid or fusaric acid. In another embodiment of the present invention, the composition comprises about 5% to about 15% of picolinic acid. Preferably, the composition comprises about 10% of picolinic acid. Unless otherwise stated herein, the percentage of a component refers to the component's percent by weight to the total weight of the composition. In still another embodiment, the composition further comprises an antibiotic, retinoid, or benzoyl peroxide.

Picolinic acid drug substance (also referred to herein as PCL-016) is an anti-infective and immunomodulator. PCL-016is a metabolite of the amino acid tryptophan. It is produced in approximately 25-50 mg quantities by the body on a daily basis by the breakdown of tryptophan, assuming normal dietary intake. PCL-016 appears to play a key role in zinc transport. As a therapeutic agent, the molecule appears to work by perturbing zinc binding in zinc finger proteins (ZFPs). ZFPs are involved in viral replication and packaging as well as normal cell homeostatic functions. Picolinic acid has been shown to be an anti-viral in vitro and in vivo, and also modifies the immune response alone and in conjunction with other cytokines such as interferon gamma. In another embodiment of the present invention, the acne vulgaris is mediated by zinc finger proteins (ZFP).

NV-02 is a PCL-016 gel product indicated for mild to moderate acne vulgaris. It is estimated that daily application of 10% PCL-016 gel would result in delivery of approximately 20 mg of PCL-016 to the surface of the skin per application. The result of a cumulative irritation study, although conducted with a different (cream) vehicle, suggested a low risk of topical irritation to the skin. An open label patient study was conducted as the first clinical evaluation of NV-02. To assess the pharmacokinetics of the gel product, plasma levels of PCL-016 were included. The patient study allows an assessment of the clinical effect of PCL-016 gel in mild to moderate acne vulgaris as well as safety information on the gel formulation.

In one embodiment, the composition reduces or inhibits total acne lesions. Preferably, the composition reduces at least about 50% of the total acne lesions. Total acne lesions comprise inflammatory and noninflammatory lesions.

In another embodiment, the composition is a topical preparation. Preferably, the topical preparation may be a cream or a gel. More preferably, the composition does not result in photosensitization or antibiotic resistance to the subject. Still preferably, the composition is non-comedogenic. That is, preferably, the composition does not contain a comedogen at a concentration that would be effective to encourage comedogenesis. More preferably, the composition is free of comedogens.

Pharmaceutically acceptable salts of picolinic acid and its derivatives may also be used, and can be prepared from pharmaceutically acceptable non-toxic acids or bases including, but not limited to, inorganic and organic acids. Buffering agents for picolinic acid or its derivatives or derivatives may also comprise non-toxic acids or bases including, but not limited to inorganic or organic acids. Examples of such inorganic acids include, but are not limited to hydrochloric, hydrobromic, hydroiodic, sulfuric and phosphoric. Organic acids may be selected, for example, from aliphatic, aromatic, carboxylic and sulfonic classes of organic acids. Examples of suitable organic acids include, but are not limited to formic, acetic, propionic, succinic, glycolic, glucoronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic and galacturonic acids. Examples of such inorganic bases for potential salt formation with the sulfate or phosphate compounds of the invention include, but are not limited to monovalent, divalent, or other metallic salts made from aluminum, calcium, lithium, magnesium, potassium, sodium and zinc. Appropriate organic bases may also be selected from N,N-dibenzylethylenediamine, chloroprocaine, choline, diethanolamine, ethylenediamine, meglumaine (N-methylglucamine), procaine, ammonia, ethylenediamine, N-methyl-glutamine, lysine, arginine, omithine, choline, N,N′-dibenzylethylenediamine, chloroprocaine, diethanolamine, procaine, N-benzylphenethylamine, diethylamine, piperazine, tris(hydroxymethyl)aminomethane and tetramethylammonium hydroxide. Carboxylic acids of picolinic acid and its derivatives are also contemplated as being within the scope of the invention.

The examples described herein provide a guidance for determining the efficacy of various formulations. Some formulations may comprise within the range of about 0.001% to about 20% of the composition, although higher concentrations may be useful in certain formulations. Preferably, the concentration of the composition ranges about 0.01% to about 10%. More preferably, the formulations comprises within about 1% to about 10% of the composition.

Depending on the particular intended use, the composition may be formulated into a variety of media such as, but not limited to, a cream, gel, ointment, lotion, paste, aerosol, solution, soap, shampoo, powder, liquid, or any other formulation capable of delivering the active agent to the affected area of a patient. The affected area may be any part of the patient's body. In one embodiment, the affected area is the skin of the face of the patient, such as the checks, nose, chin, and forehead. Other affected areas may be the skin of the back of the patient, the scalp of the patient's head, or the patient's ears.

The present invention further provides a method for treating, controlling or inhibiting acne vulgaris comprising administering to a subject afflicted with acne vulgaris a therapeutically effective amount of a composition comprising a compound having the following structure: 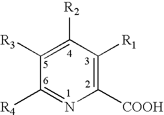
or a pharmaceutically acceptable salt thereof, wherein R₁, R₂, R₃ and R₄ are selected from a group consisting of a carboxyl group, methyl group, ethyl group, propyl group, isopropyl group, butyl group, isobutyl group, secondary butyl group, tertiary butyl group, pentyl group, isopentyl group, neopentyl group, fluorine, chlorine, bromine, iodine and hydrogen, and wherein the composition reduces or inhibits growth of Proprionibacterium acnes.

Treating or treatment, as used herein, refers to preventing a disease or symptom from occurring in an individual, inhibiting a disease or symptom from further development, or relieving the disease or symptom, resulting in regression or reversal of the disease or symptom. Also disclosed are methods of treating acne by administration of a composition comprising picolinic acid or derivatives thereof. The composition may be administered by any known means, including administration directly to the affected area.

In one embodiment, the method comprises administering to the subject the composition at least once daily. Preferably, the composition is administered to the subject twice daily. More preferably, the composition comprises about 10% picolinic acid and is administered to the subject twice daily. The composition may be administered for any number of weeks, preferably for at least about four to twelve weeks, more preferably for about 12 weeks. Still preferably, the composition may be administered for at least about 8 weeks, and even more preferably, for at least about 4 weeks.

In another embodiment, the method comprises administering the composition with a different antibiotic, retinoid, or benzoyl peroxide. Preferably, the antibiotic is a systemic preparation of tetracycline, doxycycline, metronidazole, clindamycin, erythromycin, azithromycin or minocycline, or a topical preparation of clindamycin, erythromycin, benzoyl peroxide, or metronidazole. Preferably, the retinoid is selected from the group consisting of tretinoin, adapalene, isotretinoin, or oral isotretinoin.

In still another embodiment, the method results in a reduction or inhibition of total acne lesions. Preferably, the method results in a reduction of at least about 50% of the total acne lesions. The total acne lesions comprise inflammatory and noninflammatory lesions.

In yet another embodiment, the method comprises administering to a subject a composition that is a topical preparation. Preferably, the topical preparation is a cream or gel.

In a further embodiment, the method comprises administering to a subject a composition wherein the compound has a butyl group at the R₃ position. Preferably, the compound is picolinic acid. More preferably, the composition comprises about 5% to about 15% of picolinic acid. Still more preferably, the composition comprises about 10% of picolinic acid.

In yet another embodiment, the method comprises administering to a subject the composition which further comprises propylene glycol, ethyl alcohol, hydroxyethyl cellulose, sodium chloride, and water. Preferably, the composition comprises about 10% of picolinic acid, about 5% propylene glycol, about 16% ethyl alcohol (95%), about 1% hydroxyethyl cellulose, and about 0.5% sodium chloride. It is contemplated that composition may comprise any pharmaceutically acceptable carrier. “Acceptable carrier” refers to a carrier that is compatible with the other ingredients of the formulation and is not deleterious or adverse to the recipient thereof (such as causing or increasing blackheads or whiteheads in patients). While any suitable carrier known to those of ordinary skill in the art may be employed in the pharmaceutical compositions of this invention, the type of carrier will vary depending on the mode of administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109.

In one embodiment, the composition is orally or topically administered to a subject. Preferably, the composition is administered to a mammal. More preferably, the mammal is a human. Still preferably, the administration of the composition does not result in photosensitization or antibiotic resistance to the subject and/or does not cause or increase comedones in the patient.

In another embodiment, the method treats, controls or inhibits acne vulgaris, wherein the acne vulgaris is mediated by zinc finger proteins (ZFP).

In an embodiment, the method provides a prophylactic treatment of acne vulgaris. As described in Example 3 herein, subjects may have a high level of colonization of P. acnes, but may not have developed acne yet. The example demonstrates that the method described herein can reduce or inhibit the colonization of P. acnes, thereby preventing acne from developing.

### EXAMPLES

### Example 1

### Antimicrobial Susceptibility Testing of Propionibacterium acnes in Picolinic and Fusaric Acids by the Agar Dilution Method.

The purpose of this study was to determine the Minimum Inhibitory Concentration (MIC) of Picolinic acid and fusaric acid that would visibly inhibit the growth of Propionibacterium acnes. The procedure for this study was based on “NCCLS Methods for Antimicrobial Susceptibility Testing of Anaerobic Bacteria; Approved Standard—Fifth edition” and used the agar dilution method.

Picolinic acid and fusaric acid were a white crysalline material and a white powder, respectively. Stock solutions of the test substances were prepared the day of testing in ABC reagent water that had been autoclaved for sterilization. A stock solution of Picolinic acid was prepared by adding 2.010 g of the test substance to 200 mL of ABC reagent water to make a concentration of 10 mg/mL. The stock solution of Fusaric acid was prepared by adding 0.5000 g of test substance to 100 mL of ABC reagent water to make a concentration of 5.0 mg/mL. Dilutions from the stock solution of Picolinic acid were performed to make working standards with the following concentrations: 5, 1, 0.5, 0.1, 0.05, and 0.01 mg/mL. Dilutions from the stock solution of Fusaric acid were performed to make working standards with the following concentrations: 1, 0.5, 0.1, 0.05, and 0.01 mg/mL. All stock solutions and working standards were made using autoclaved ABC reagent water and autoclaved glassware to prevent microbial contamination.

The organism tested was Propionibacterium acnes ATCC #11827. This organism was received from the American Type Culture Collection (ATCC). Propionibacterium acnes is a gram positive, pleomorphic, anaerobic to aerotolerant rod. Cultures were maintained in 20% glycerol in a −80° C. freezer. Prior to conducting a test, the organism was subcultured from frozen stock three times on Supplemented Brucella Agar with blood. A gram stain was performed to check the identity of the culture. Cultures were incubated in anaerobic gas jars at 35±2° C.

Agar Preparation

Supplemented Brucella agar was prepared and dispensed in 17.4 mL volumes into 50×150 mm test tubes. These tubes were then autoclaved and the volume after autoclaving was approximately 17 mL. The tubes were stored under refrigeration until test initiation.

Test Plate Preparation

On test initiation, the Supplemented Brucella agar tubes were flash autoclaved for approximately 5 minutes to melt the agar and placed in a ˜45° C. water bath to cool. A 2-mL volume of working standard or stock solution and a1-ml volume of defibrinated sheep's blood were added to each tube. Each tube was inverted to mix and the contents were poured into a sterile, labeled petri plate. The final volume of each test plate was 20 mL. Duplicate plates were prepared at each test concentration. Test article concentrations in the agar media were 0.0 (control), 0.001, 0.005, 0.01, 0.05, 0.1, 0.5 and 1 mg/mL for Picolinic acid. Fusaric acid test article concentrations were 0.0 (control), 0.001, 0.005, 0.01, 0.05, 0.1, and 0.5 mg/mL in the agar media.

Inoculation of Test Plates

P. acnes was inoculated onto the test plates after the agar media solidified. A broth culture was prepared as the inoculum source. The broth culture was prepared by adding solid growth from a third serial subculture of the frozen stock to 5 mL of Brucella broth. P. acnes colonies were added till the broth visually matched the density of a 0.5 McFarland Standard. The inoculum was “spotted” onto eight sections of the plate, making eight spots per plate. Inoculum was applied using a Rainin lus pipetter set to draw 100 pL and dispense in 2 p.L drops.

Before inoculation began, two test plates were inoculated and labeled “pre-Ana” and “pre-Oz”. This was repeated between test article plate sets and after all inoculation had been performed to make “mid-Ana”, “mid-02” and “post-Ana”, “Post 02” plates, respectively. The “Ana” plates were prepared as an anaerobic growth control to assure culture viability. The “02” plates were prepared to check for aerobic contamination.

All plates were then incubated until growth was observed on the control plates.

Incubation of Test Plates

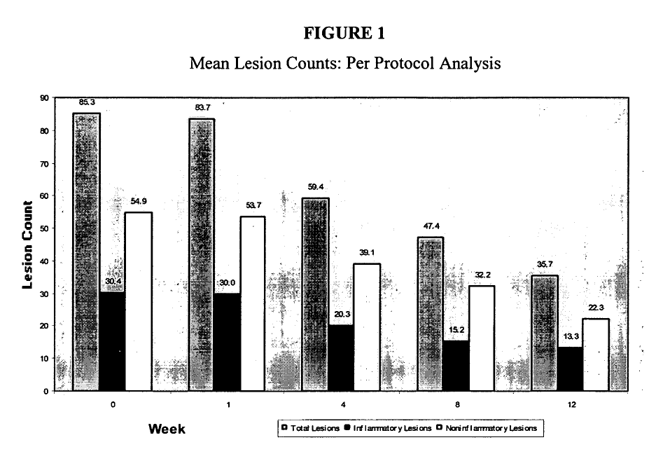

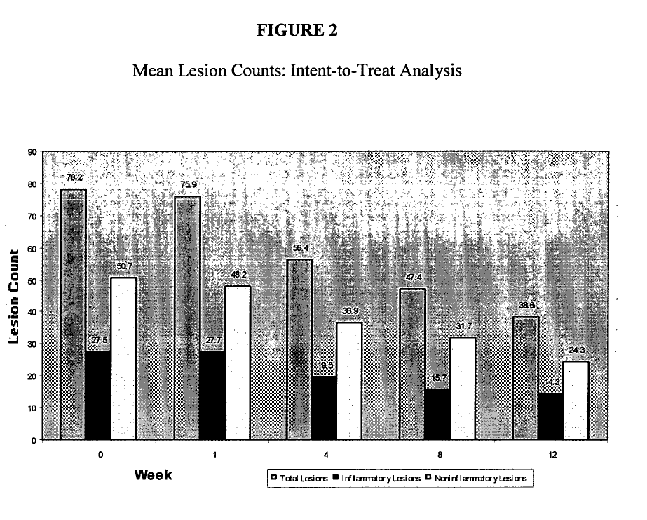

All test plates and anaerobic controls were incubated for 48-72 hours at 35±2° C. in an anaerobic gas jar. All aerobic controls were incubated for 48-72 hours at 35±2° C. under aerobic conditions. After the incubation period, the number of spots with growth was documented. Selected plates were digitally photographed (FIGS. 1 and 2).

Inoculum Control

Microbial plate counts were performed at test initiation on the 5-mL broth inoculum used for test plate inoculation. The McFarland standard used to adjust the inoculum would give approximately 1.5×108 CFU/mL.

Determination of Minimum Inhibitory Concentration

The minimum inhibitory concentration (MIC) of each test article was determined for P. acnes. This value was defined as the lowest concentration of the test article that was visibly observed to completely inhibit the growth of the organism. The MIC endpoint occurs where there is a marked reduction in the appearance of growth on the test plate as compared to that of growth on the control plate. (Table 1 and 2)

Results

Microbial plate counts showed that 2.7×109 CFU/mL was the viable count on the broth used for spot inoculation. This was a sufficient amount of inoculum to spot inoculate the test plates. Growth on the “Ana” control plates indicated that the culture remained viable throughout the test plate inoculation. No growth on the “O” plates indicated that the culture was not contaminated with aerobic organisms. Furthermore, growth on the “Ana” plates matched the typical colony morphology and gram stain morphology for P. acnes. The lowest concentration of Picolinic acid that visibly prevented growth was 0.5 mg/mL. The lowest concentration of fusaric acid that visibly prevented growth was 0.1 mg/mL.

### Example 2

Example 2 demonstrates the safety and the potential efficacy of NV-02 in the topical treatment of mild to moderate acne vulgaris.
Name of Finished Product: 10% Picolinic Acid Gel (NV-02)
Name of Active Ingredient: Picolinic Acid (PCL-016)
Study period: 3 months
Date of first enrollment: Oct. 22, 2002
Date of last completed: May 2, 2003
Methodology: Open label study
Number of patients (planned and analyzed): 15 patients planned, 20 enrolled, 15 completed.
Diagnosis and main criteria for inclusion: mild to moderate acne vulgaris
Test product, dose and mode of administration, batch number: NV-02 applied twice daily to affected areas of the face; Novactyl lot number 155
Duration of treatment: twice daily for 3 months
Criteria for evaluation:
   Efficacy: Total lesion counts, inflammatory lesion count, non-inflammatory lesion count, and Cunliffe's grade, FDA scale
   Safety: Adverse events, PCL-0 16 plasma levels, serum chemistry, complete blood count
Statistical Methods: descriptive statistics of patient population, patient demographics, and lesion counts, etc.; changes in lesion counts relative to baseline; percent reduction in lesion counts.

Results

NV-02 applied topically twice daily to affected areas of the face was well tolerated in this study. One patient, however, experienced burning on application of NV-02 to acne lesions on the face. There were no other adverse events in this study. Serial complete blood counts and serum chemistries did not reveal bone marrow, liver, or renal toxicity. Small amounts of PCL-016 were absorbed into the systemic circulation. Absorption tended to vary between patients and within patients.

Efficacy Results:

NV-02 reduced the mean total lesion count, mean inflammatory lesion count and mean non-inflammatory lesion count by 54.3%, 51.4%, and 56% respectively in patients in this study. The changes in the mean total lesion count, mean inflammatory lesion count, and mean non-inflammatory lesion count compared to baseline were statistically significant (p=0.0006, p=0.001, and p=0.006 respectively).

List of Abbreviations and Definitions:

NV-02 =test product. Used synonymously with 10% PCL-016 gel.
PCL-0 16=Picolinic acid drug substance
Zinc Finger Protein=ZFP

Statistical Output
Obs.=observations
Std. Dev.=standard deviation
Std. err.=standard error of the mean
Conf. interval=confidence interval
Infl=Inflammatory lesion count
NonInfl=Non-inflammatory lesion count
Pt=patient

Preparation of the Anti-Acne Composition

The investigational test product utilized in this study was NV-02, a gel formulation consisting of picolinic acid, propylene glycol (USP), ethyl alcohol (95%), hydroxyethyl cellulose (NF), sodium chloride (USP), and water. NV-02 is packaged in 6 g high density polyethylene COEX tubes. The corresponding Novactyl product lot number is 155. The product has remained physically and chemically stable when stored at 25 C/60% RH for up to 36 months.

TABLE 1

| | | Unit (6 g tube) | 10 kg Batch |
|---|---|---|---|
| Component | Percentage | Formulation, g | Formulation, g |
| Picolinic acid | 10.0 | 0.6 | 1000 |
| Propylene Glycol, USP | 5.2 | 0.31 | 518 |
| Ethyl Alcohol, 95% | 16.2 | 0.97 | 1620 |
| Hydroxylethyl Celluolose, | 1.0 | 0.06 | 100 |
| NF | | | |
| Sodium Chloride, USP | 0.5 | 0.03 | 50 |
| Sterile Water for Irrigation, | 67.1 | 4.03 | 6712 |
| USP | | | |
| Total (approximate) | 100 | 6 | 10,000 |

Hypothesis Testing: Comparisons of Lesion Counts at Week 12 Relative to Baseline For All Patients.

Test of Two Means: Total Lesions at Week 0 vs. Week 12 For All Patients.

Two-Sample T Test with Equal Variances

| | Obs | Mean | Std. Err. | Std. Dev. | [95% Conf. Interval] | |
|---|---|---|---|---|---|---|
| x | 20 | 78.15 | 9.219308 | 41.23 | 58.85377 | 97.44623 |
| y | 15 | 35.67 | 4.084706 | 15.82 | 26.90918 | 44.43082 |
| com- | 35 | 59.94429 | 6.563826 | 38.83212 | 46.60499 | 73.28358 |
| bined | | | | | | |
| diff | 42.48 | 11.25048 | 19.59073 | 65.36927 | | |

Degrees of freedom: 33
Ho: mean(x) − mean(y) = diff = 0

| | | |
|---|---|---|
| Ha: diff < 0 | Ha: diff != 0 | Ha: diff > 0 |
| t = 3.7758 | t = 3.7758 | t = 3.7758 |
| P < t = 0.9997 | P > t = 0.0006 | P > t = 0.0003 |

Test of Two Means: Inflammatory Lesions at Week 0 vs. Week 12 For All Patients.

Two-Sample T Test with Equal Variances

| | Obs | Mean | Std. Err. | Std. Dev. | [95% Conf. Interval] | |
|---|---|---|---|---|---|---|
| x | 20 | 27.45 | 3.101426 | 13.87 | 20.95864 | 33.94136 |
| Y | 15 | 13.33 | 1.77899 | 6.89 | 9.514445 | 17.14555 |
| com- | 35 | 21.39857 | 2.250806 | 13.31595 | 16.82438 | 25.97276 |
| bined | | | | | | |
| diff | 14.12 | 3.90793 | 6.169257 | 22.07074 | | |

Degrees of freedom: 33
Ho: mean(x) − mean(y) = diff = 0

| | | |
|---|---|---|
| Ha: diff < 0 | Ha: diff != 0 | Ha: diff > 0 |
| t = 3.6132 | t = 3.6132 | t = 3.6132 |
| P < t = 0.9995 | P > t = 0.0010 | P > t = 0.0005 |

Test of Two Means: Non-Inflammatory Lesions at Week 0 vs. Week 12

Two-Sample T Test with Equal Variances

| | Obs | Mean | Std. Err. | Std. Dev. | [95% Conf. Interval] | |
|---|---|---|---|---|---|---|
| x | 20 | 50.7 | 8.045373 | 35.98 | 33.86084 | 67.53916 |
| Y | 15 | 22.33 | 2.912483 | 11.28 | 16.08334 | 28.57666 |
| com- | 35 | 38.54143 | 5.288068 | 31.28463 | 27.79478 | 49.28808 |
| bined | | | | | | |
| diff | 28.37 | 9.656889 | 8.722912 | 48.01709 | | |

Degrees of freedom: 33
Ho: mean(x) − mean(y) = diff = 0

| | | |
|---|---|---|
| Ha: diff < 0 | Ha: diff != 0 | Ha: diff > 0 |
| t = 2.9378 | t = 2.9378 | t = 2.9378 |
| P < t = 0.9970 | P > t = 0.0060 | P > t = 0.0030 |

Percent Reductions in Lesion Counts Relative to Baseline For All Patients.

The following table summarizes means in lesion counts by week.

TABLE 2

| Week | Total | Infl | Nonlnfl |
|---|---|---|---|
| −1 | 92.625 | 30.3125 | 62.3125 |
| 0 | 78.15 | 27.45 | 50.7 |
| 1 | 77.3158 | 28.3684 | 48.9474 |
| 4 | 56.6471 | 19.5882 | 37.0588 |
| 8 | 47.4 | 15.2 | 32.2 |
| 12 | 35.6667 | 13.3333 | 22.3333 |

During the twelve week study, the mean total lesion count was reduced by 54.3%. The mean inflammatory lesion count was reduced by 51.4%. The mean non-inflammatory lesion count was reduced by 56.0%.

### Example 3

It is widely accepted that the ability of a test product to produce reductions in P. acnes colony counts on the skin of healthy volunteers with little to no acne reliably predicts the ability of that test product to reduce P. acnes colony counts and treat acne in patients with acne. (Leyden, James L., The Evolving Role of Proprionibacterium Acnes in Acne, SEMINARS IN CUTANEOUS MEDICINE AND SURGERY, 20(3):139-143, September 2001.) Twenty normal, healthy adult males and females between the ages of 19 and 53 years were selected to evaluate NV-02 for its ability to reduce P. acnes colony counts on the skin. The volunteers who were selected for the study were essentially free of acne but had a high degree of fluorescence of the facial skin under a Wood's lamp examination indicating the presence of high levels of P. acnes. They were carefully screened to ensure that none were using any form of topical or systemic antibiotics within 4 weeks prior to enrollment. They were given a non-antimicrobial soap (Dove) provided by the testing laboratory to use throughout the study and were instructed not to use any medicated shampoos. Each subject was also given an instructional sheet which specified products to be avoided, scheduled laboratory visits for supervised product applications and instructions on how to apply the product at night to the test area (forehead).

The following subjects were excluded from the study:
Volunteer who exhibited any skin disorders of an acute or chronic nature including psoriasis, eczema, etc.
Use of topical or systemic antibiotics within the previous 4 weeks
Use of other medications which would have influenced skin surface or P. acnes levels (e.g., retinoids, antibiotics and corticosteroids) within the previous 6 months
History of any significant internal disease
Female who were pregnant, planning a pregnancy or breastfeeding
Subjects who were known to be allergic to any of the test product(s) or any components in the test product(s)
Past or present history of drug abuse
AIDS or AIDS Related Complex
Any subject not able to meet the study attendance requirements

Treatment Plan:

Treatment with NV-02 (PCL-016 gel) was applied for 4 weeks. Each volunteer was treated once daily under supervision by a technician at Ivy Laboratories in a standardized manner beginning on the Wednesday following baseline testing and evaluation for acceptance into the study. The test product was also applied by the subjects (unsupervised) at home once daily before bedtime and twice on Saturdays and Sundays. No concurrent therapy of any kind or topical and/or systemic antibacterial agent were allowed during the course of the study. At each visit, a sufficient amount of the test product (about 0.5 ml) was applied to the entire forehead area and rubbed in for about 30 seconds. A bland unmedicated soap (Dove) was given to the volunteers for the purposes of washing, showering and bathing.

Quantitative Bacteriology:

Quantitative bacteriologic cultures were obtained from the test site (forehead) at baseline, two weeks and four weeks. Samples were obtained according to the technique of Williamson and Kligman (scrub procedure for obtaining P. acnes samples). One side of the forehead was cleansed of surface bacteria by thoroughly wiping the area for 30 seconds with sterile gauze soaked with 0.1% Triton-X-100 to remove surface debris and bacteria. The area to be scrubbed (3.8 cm²) was delineated by a sterile glass cylinder held firmly to the skin. One ml of wash solution [Bacto Letheen Broth, Difco] was pipetted into the cylinder and the area scrubbed with moderate pressure for one minute using a sterile Teflon “Policeman”. The wash fluid was aspirated, replace with a fresh 1 ml and the scrub repeated. Thus, the skin was sampled using two 1 ml quantities of Bacto Letheen Broth. The 2 mL skin surface scrub was serially diluted into 0.05% Tween-80 (buffered with 0.075M phosphate buffer, pH 7.9) in 4 ten-fold dilutions. Using a micropipettor, 50 mL of each dilution was placed on a designated section of an agar plate containing Brucella agar supplemented with yeast extract, dextrose, and cysteine, five drop dilutions per plate. Plates were allowed to dry, placed in an anaerobic jar with BBL Gas Pak Plus anaerobic system envelope and incubated anaerobically at 35-35° C. for 7 days. Colony forming units (cfu) of P. acnes were counted at the dilution that contains between 10 and 100 cfu. Total densities of P. acnes were calculated and reported as log₁₀ cfu per cm².

Results:

A total of 20 subjects who qualified based on the baseline P. acnes levels were enrolled into the study. There were 18 females and 2 males ranging in age from 19 to 53 years. All 20 subjects completed the investigation. There were no adverse reactions noted in any, and no unexpected reactions were seen throughout the study period.

The results of this in-vivo assay of the antibacterial effect on P. acnes colonization of sebaceous follicle shows that the test drug produced a significant reduction in the levels of P. acnes as early as 2 weeks after treatment. (Table 3). This reduction was maintained and somewhat extended after an additional 2 weeks of treatment. Following the first 2 weeks of treatment, there was a mean reduction of 1.0 log and by 4 weeks, the mean reduction was still 1.0 log. Using a paired T-Test, the reduction at both 2 weeks and at 4 weeks was highly significant (P<0.001). Very meaningful is the consistency of reduction seen in this panel. This suggests that lower concentrations may also produce a significant reduction in P. acnes levels.

The results exceed those observed with the antibiotics clindamycin and erythromycin. Since it is known that the pharmacodynamic effect of P. acnes reduction is the primary mechanism of action for antibiotic therapy in acne, the results indicate that this agent (NV-02, 10% PCL-016 gel) will be effective in treating acne.

TABLE 3

| SCR | | Patient | First | Last | Baseline | Week2 | Week 4 |
|---|---|---|---|---|---|---|---|
| # | Study # | ID | Name | Name | log/cm² | log/cm² | log/cm² |
| 13 | 1 | H268 | | | 6.69 | 6.29 | 5.54 |
| 4 | 2 | S186 | | | 6.79 | 4.95 | 5.15 |
| 2 | 3 | R10 | | | 5.35 | 4.99 | 4.15 |
| 9 | 4 | P138 | | | 7.31 | 6.32 | 6.25 |
| 5 | 5 | T042 | | | 6.11 | 5.43 | 4.45 |
| 10 | 6 | B067 | | | 7.00 | 5.94 | 5.84 |
| 20 | 7 | F203 | | | 6.57 | 5.23 | 4.15 |
| 6 | 8 | H241 | | | 7.01 | 6.08 | 5.95 |
| 3 | 9 | C383 | | | 7.09 | 6.16 | 6.04 |
| 7 | 10 | H199 | | | 5.95 | 4.69 | 4.45 |
| 16 | 11 | B411 | | | 7.19 | 6.50 | 6.41 |
| 17 | 12 | M349 | | | 6.98 | 4.81 | 4.30 |
| 1 | 13 | P052 | | | 6.89 | 6.07 | 5.45 |
| 14 | 14 | C326 | | | 6.99 | 5.84 | 4.89 |
| 11 | 15 | N007 | | | 7.09 | 6.24 | 5.87 |
| 18 | 16 | D379 | | | 6.95 | 4.97 | 5.65 |
| 21 | 17 | C290 | | | 6.81 | 4.69 | 4.65 |
| 8 | 18 | F164 | | | 7.22 | 6.00 | 5.69 |
| 19 | 19 | K207 | | | 5.66 | 2.06 | 5.15 |
| 15 | 20 | G119 | | | 7.20 | 4.85 | 4.24 |

| | | | |
|---|---|---|---|
| Mean | 6.741 | 5.405 | 5.214 |
| Std | 0.548 | 1.007 | 0.754 |
| Dev | | | |
| Paired | | 0.0000 | 0.0000 |
| T-Test | | | |

References
1. American Type Culture Collection (ATCC). 1992. Catalogue of Bacteria and Bacteriophages, 18^{th} edition, Rockville, Md.
2. Blasi, E. et al., Protective Effect of Picolinic Acid on Mice Intracerebrally Infected with Lethal Doses of Candida albicans, ANTIMICROBIAL AGENTS AND CHEMOTHERAPY, 37(11):2422-2426, November 1993.
3. Collins J. J. et al., Transient growth inhibition of Escherichia coli K-12 by ion chelators: “in vivo” inhibition of ribonucleic acid synthesis, J. BACTERIOL., 138(3):923-932, 1979.
4. Fortnagel, P. and E. Freese, Inhibition of aconitase by chelation of transition metals causing inhibition of sporulation in Bacillus subtilis, J. BIOL. CHEM., 243(20):5289-5295, Oct. 25, 1968.
5. Evans, G. W., and P. E. Johnson, Characterization and quantitation of a zinc-binding ligand in human milk. PEDIATRI. RES., 14:876-880, 1980.
6. Fernandez-Pol, J. A., Transition metal ions induce cell growth in NRK cells synchronized in G1 by picolinic acid, BIOCHEM. BIOPHYS. RES. CUMMUN., 76:413-419, 1977.
7. Fernandez-Pol J A. Iron: possible cause of the G₁ arrest induced in NRK cells by picolinic acid. BIOCHEM. BIOPHYS. RES. COMMUN., 78(1):136-143, Sep. 9, 1977.
8. Johnson, G. S. and J. A. Fernandez-Pol, NRK cells synchronized in G₁ by picolinic acid are super-sensitive to prostaglandin E1 stimulation, FEBS. LETT., 74(2):201-204, Mar. 1, 1977
9. Leyden, J. J., McGinley, K. J. and Kligman, A. M.: Tetracycline and minocycline treatment. Effects on skin-surface lipid levels and propionibacterium acnes. Arch. Dermatol., Vol. 118, January 1982.
10. Leyden, James L., The Evolving Role of Proprionibacterium Acnes in Acne, SEMINARS IN CUTANEOUS MEDICINE AND SURGERY, 20(3):139-143, September 2001.
11. NCCLS. Methods for Antimicrobial Susceptibility Testing of Anaerobic Bacteria; Approved Standard -Fifth edition. NCCLS document M 11-A5 (ISBN 1-56238-429-5). NCCLS, 940 West Valley Road, Suite 1400, Wayne, Pa.
12. R. E. Buchanan & N. E. Gibbons. Bergey's manual of determinative bacteriology, 8th ed. Williams & Wilkins Co. 1974. Baltimore, Md.
13. Ruffmann R, et al., In vivo activation of macrophages but not natural killer cells by picolinic acid (PLA), J. IMMUNOPHARMACOL., 6(4):291-304, 1984.
14. Ruggieri, M. R., McGinley, K. J. and Leyden, J. J.: Reproducibility and precision of quantitation of skin surface lipds by thin layer chromatography (Chapter 19) in: Advanced in thin layer chromatography Edited by Touchstone, J. C., Wiley Press, pp. 249-259, 1982.
15. Varesio L. et al., Picolinic acid, a catabolite of tryptophan, as the second signal in the activation of IFN-gamma-primed macrophages, J. IMMUNOL., 145(12):4265-427 1, Dec. 15, 1990.
16. Williamson, P. and Kligman, A. M.: A new method for the quantitative investigation of cutaneous bacteria. Journal of Investigative Dermatology 45: 498-503, 1965.
17. Nordstrom, K. M., Schmus, H. G., McGinley, K. J. and Leyden, J. J.: Measurement of seburm output using a lipid absorbent tape. The Society for Investigative Dermatology, Inc., Vol. 87 No. 2, August 1986.

1. A method for treating, controlling or inhibiting acne vulgaris comprising administering to a subject afflicted with acne vulgaris a therapeutically effective amount of a composition comprising a compound having the following structure: 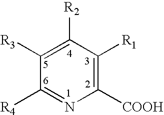
or a pharmaceutically acceptable salt thereof,
wherein R₁, R₂, R₃ and R₄ are selected from a group consisting of a carboxyl group, methyl group, ethyl group, propyl group, isopropyl group, butyl group, isobutyl group, secondary butyl group, tertiary butyl group, pentyl group, isopentyl group, neopentyl group, fluorine, chlorine, bromine, iodine and hydrogen, and
wherein the composition reduces or inhibits growth of Proprionibacterium acnes.

2. The method of claim 1, wherein administration of the composition to the subject provides a prophylactic treatment of acne vulgaris.

3. The method of claim 1, wherein the composition further comprises an antibiotic, retinoid, or benzoyl peroxide.

4. The method of claim 3, wherein the antibiotic is a systemic preparation of tetracycline, doxycycline, metronidazole, clindamycin, erythromycin, azithromycin or minocycline, or a topical preparation of clindamycin, erythromycin, benzoyl peroxide, or metronidazole.

5. The method of claim 3, wherein the retinoid is selected from the group consisting of tretinoin, adapalene, isotretinoin, or oral isotretinoin.

6. The method of claim 1, wherein the composition reduces or inhibits total acne lesions.

7. The method of claim 6, wherein the composition reduces at least about 50% of the total acne lesions.

8. The method of claim 6, wherein the total acne lesions comprise inflammatory and noninflammatory lessions.

9. The method of claim 1, wherein the composition is a topical preparation and the composition is administered to the subject by topical application.

10. The method of claim 9, wherein the topical preparation is a cream.

11. The method of claim 9, wherein the topical preparation is a gel.

12. The method of claim 1, wherein the administration of the composition does not result in photosensitization or antibiotic resistance to the subject.

13. The method of claim 12, wherein the subject is a mammal.

14. The method of claim 13, wherein the mammal is a human.

15. The method of claim 1, wherein R₃ is butyl.

16. The method of claim 15, wherein the compound is picolinic acid or a derivative thereof.

17. The method of claim 16, wherein the composition comprises about 5% to about 15% of picolinic acid.

18. The method of claim 17, wherein the composition is administered to the subject at least once daily.

19. The method of claim 18, wherein the composition is administered to the subject twice daily.

20. The method of claim 17, wherein the composition comprises about 10% of picolinic acid.

21. The method of claim 20, wherein the composition is administered to the subject at least once daily.

22. The method of claim 21, wherein the composition is administered to the subject twice daily.

23. The method of claim 1, wherein the composition further comprises propylene glycol, ethyl alcohol, hydroxyethyl cellulose, sodium chloride, and water.

24. The method of claim 1, wherein the composition is orally or topically administered to a subject.

25. The method of claim 24, wherein the subject is a mammal.

26. The method of claim 25, wherein the mammal is a human.

27. The method of claim 1, wherein the acne vulgaris is mediated by zinc finger proteins (ZFP).

28. A pharmaceutical composition comprising a compound having the following structure: 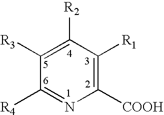
or a pharmaceutically acceptable salt thereof,
wherein R₁, R₂, R₃ and R₄ are selected from a group consisting of a carboxyl group, methyl group, ethyl group, propyl group, isopropyl group, butyl group, isobutyl group, secondary butyl group, tertiary butyl group, pentyl group, isopentyl group, neopentyl group, fluorine, chlorine, bromine, iodine and hydrogen, and
wherein the amount of the compound in the pharmaceutical composition is sufficient to reduce or inhibit growth of Proprionibacterium acnes by at least 1 or 2 logarithm.

29. The pharmaceutical composition of claim 28, further comprising an antibiotic, retinoid, or benzoyl peroxide.

30. The pharmaceutical composition of claim 29, wherein the antibiotic is a systemic preparation of tetracycline, doxycycline, metronidazole, clindamycin, erythromycin, azithromycin or minocycline, or a topical preparation of clindamycin, erythromycin, benzoyl peroxide, or metronidazole.

31. The pharmaceutical composition of claim 29, wherein the retinoid is selected from the group consisting of tretinoin, adapalene, isotretinoin, or oral isotretinoin.

32. The pharmaceutical composition of claim 28, wherein the composition comprises about 5% to about 15% of the compound, propylene glycol, ethyl alcohol, hydroxyethyl cellulose, and sodium chloride.

33. The pharmaceutical composition of claim 32, wherein the pharmaceutical composition comprises about 10% of picolinic acid, about 5% propylene glycol, about 16% ethyl alcohol (95%), about 1% hydroxyethyl cellulose, and about 0.5% sodium chloride.

34. The pharmaceutical composition of claim 28, wherein the compound is picolinic acid.

35. The pharmaceutical composition of claim 29, wherein the compound is picolinic acid.

36. The pharmaceutical composition of claim 28, wherein the composition reduces at least about 50% of total acne lesions in a subject afflicted with acne vulgaris.

37. The pharmaceutical composition of claim 36, wherein the total acne lesions comprise inflammatory and noninflammatory lessions.

38. The pharmaceutical composition of claim 28, wherein the pharmaceutical composition is a topical preparation and the composition is administered to the subject by topical application.

39. The pharmaceutical composition of claim 38, wherein the topical preparation is a cream.

40. The pharmaceutical composition of claim 38, wherein the topical preparation is a gel.

41. The pharmaceutical composition of claim 28, wherein the pharmaceutical composition does not cause photosensitization or antibiotic resistance in the subject.

42. The pharmaceutical composition of claim 28, wherein the pharmaceutical composition is non-comedogenic.
